(19) United States
(12) Patent Application Publication
(10) Pub. No.: US 2019/0284158 A1
(43) Pub. Date: Sep. 19, 2019

(54) 4-((6-(2-(2,4-DIFLUOROPHENYL)-1,1-DIFLUORO-2-HYDROXY-3-(5-MERCAPTO-1H-1,2,4-TRIAZOL-1-YL)PROPYL)PYRIDIN-3-YL)OXY)BENZONITRILE AND PROCESSES OF PREPARATION

(71) Applicant: Dow AgroSciences LLC, Indianapolis, IN (US)

(72) Inventors: Kaitlyn Gray, Indianapolis, IN (US); Qiang Yang, Zionsville, IN (US); Nicholas R. Babij, Indianapolis, IN (US); Yan Hao, Zionsville, IN (US); Jim Renga, Spokane, WA (US)

(21) Appl. No.: 16/462,202

(22) PCT Filed: Nov. 17, 2017
(86) PCT No.: PCT/US2017/062143

§ 371 Date: May 17, 2019

Related U.S. Application Data
(60) Provisional application No. 62/423,862, filed on Nov. 18, 2016.

(51) Int. Cl. C07D 401/06; C07D 213/65

(52) U.S. Cl. CPC C07D 401/06; C07D 213/65

(57) ABSTRACT

Provided herein is a process for the preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile.

### CROSS-REFERENCE TO RELATED APPLICATIONS

The present application claims priority under 35 U.S.C. § 119(e) to U.S. provisional patent application, U.S. Ser. No. 62/423,862, filed Nov. 18, 2016, the entire contents of which is incorporated herein by reference.

### FIELD

Provided herein is 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile and processes of preparation.

### BACKGROUND

U.S. Patent Application Ser. No. 62/163,106 describes inter alia certain metalloenzyme inhibitor compounds and their use as fungicides. The disclosure of this application is expressly incorporated by reference herein. This patent application describes various routes to generate metalloenzyme inhibiting fungicides. It may be advantageous to provide more direct and efficient methods for the preparation of metalloenzyme inhibiting fungicides and related compounds, e.g., by the use of reagents and/or chemical intermediates which provide improved time and cost efficiency.

### SUMMARY OF THE DISCLOSURE

Provided herein is the compound 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) and processes for its preparation. In one embodiment, provided herein, is a process for the preparation of the compound of the Formula I:

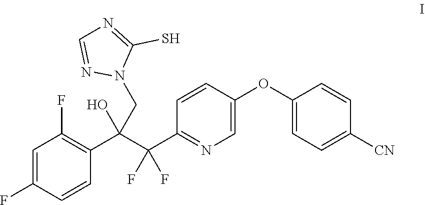 [SMILES: N#Cc1ccc(Oc2ccc(C(F)(F)C(O)(Cn3ncnc3S)c3ccc(F)cc3F)nc2)cc1]

which comprises contacting a compound of Formula II with a trialkyl orthoformate.

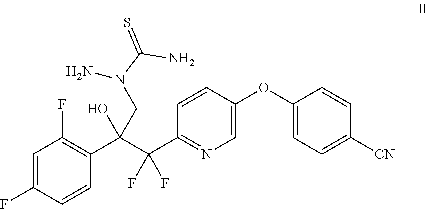 [SMILES: N#Cc1ccc(Oc2ccc(C(F)(F)C(O)(CN(N)C(N)=S)c3ccc(F)cc3F)nc2)cc1]

In one embodiment, the compound of Formula II may be prepared by contacting a compound of Formula III with an acid.

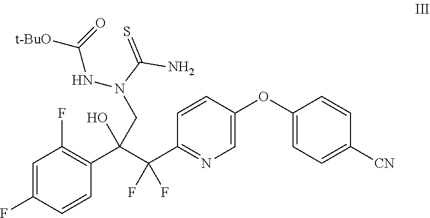 [SMILES: CC(C)(C)OC(=O)NN(CC(O)(c1ccc(F)cc1F)C(F)(F)c1ccc(Oc2ccc(C#N)cc2)cn1)C(N)=S]

Another aspect of the present disclosure is the novel intermediate produced in the present process, viz., a compound consisting of:

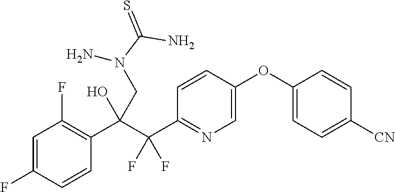 [SMILES: N#Cc1ccc(Oc2ccc(C(F)(F)C(O)(CN(N)C(N)=S)c3ccc(F)cc3F)nc2)cc1]

The term “halogen” or “halo” refers to one or more halogen atoms, defined as F, Cl, Br, and I.

The term “organometallic” refers to an organic compound containing a metal, especially a compound in which a metal atom is bonded directly to a carbon atom.

Room temperature (RT) is defined herein as about 20° C. to about 25° C.

Throughout the disclosure, references to the compounds of Formula I-III are read as also including optical isomers and salts. Specifically, when compounds of Formula I-III contain a chiral carbon, it is understood that such compounds include optical isomers and racemates thereof. Exemplary salts may include: hydrochloride salts, hydrobromide salts, hydroiodide salts, and the like.

Certain compounds disclosed in this document can exist as one or more isomers. It will be appreciated by those skilled in the art that one isomer may be more active than the others. The structures disclosed in the present disclosure are drawn in only one geometric form for clarity, but are intended to represent all geometric and tautomeric forms of the molecule. For example, the chemical structures of Formulas I and Ia are tautomeric forms of the same molecule.

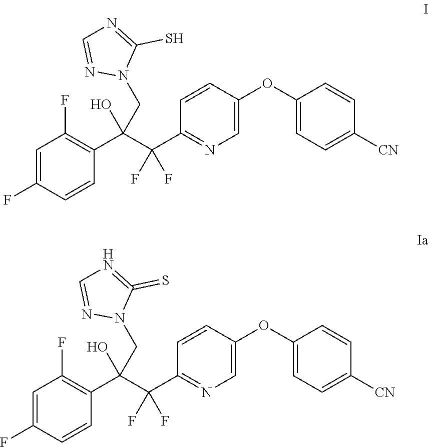

The embodiments described above are intended merely to be exemplary, and those skilled in the art will recognize, or will be able to ascertain using no more than routine experimentation, numerous equivalents of specific processes, materials and procedures. All such equivalents are considered to be within the scope of the invention and are encompassed by the appended claims.

### DETAILED DESCRIPTION

4-((6-(2-(2,4-Difluorophenyl)-1,1-difluoro-2-hydroxy-3-(5-mercapto-1H-1,2,4-triazol-1-yl)propyl)pyridin-3-yl)oxy)benzonitrile (I) is provided herein and may be prepared from 1-(3-(5-(4-cyanophenoxy)pyridin-2-yl)-2-(2,4-difluorophenyl)-3,3-difluoro-2-hydroxypropyl)hydrazine-1-carbothioamide (II) as shown in Example 1.

### Example 1: Preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I)

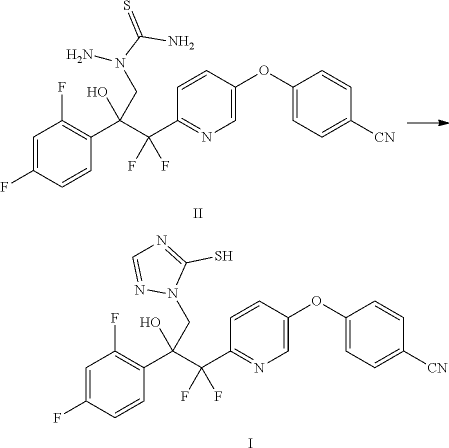

1A: To 1-(3-(5-(4-cyanophenoxy)pyridin-2-yl)-2-(2,4-difluorophenyl)-3,3-difluoro-2-hydroxypropyl)hydrazine-1-carbothioamide (II, 0.46 g, 0.936 mmol) was added triethyl orthoformate (3.12 mL, 18.72 mmol) and the reaction mixture was heated at 100° C. for 5 h. The reaction was cooled to RT, diluted with ethyl acetate, and washed with saturated ammonium chloride followed by saturated sodium bicarbonate. The organic layer was dried over anhydrous sodium sulfate, filtered, and concentrated. The crude material was dissolved in dichloromethane and purified by silica gel column chromatography with 0-50% ethyl acetate/hexanes as the eluent. Product containing fractions were collected and concentrated giving 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) as a white foam (327.6 mg, 0.653 mmol, 70% yield). ¹H NMR (400 MHz, CDCl₃) δ 8.44 (d, J=2.7 Hz, 1H), 7.72-7.65 (m, 2H), 7.62 (s, 1H), 7.58 (d, J=8.6 Hz, 1H), 7.50-7.36 (m, 2H), 7.10-7.02 (m, 2H), 6.80 (ddd, J=11.5, 8.6, 2.6 Hz, 1H), 6.76-6.69 (m, 1H), 5.93 (s, 1H), 5.31-5.21 (m, 2H). ¹⁹F NMR (376 MHz, CDCl₃) δ−103.15 (ddd, J=31.2, 23.4, 9.4 Hz),−108.46 (d, J=29.1 Hz),−109.02 (d, J=23.2 Hz),−109.39 (d, J=9.2 Hz). ESIMS m/z 502.0 [(M+H)⁺].

1B: To 1-(3-(5-(4-cyanophenoxy)pyridin-2-yl)-2-(2,4-difluorophenyl)-3,3-difluoro-2-hydroxypropyl)hydrazine-1-carbothioamide (0.25 g, 0.509 mmol) in acetonitrile (2.5 mL) was added triethyl orthoformate (0.212 mL, 1.272 mmol) and the reaction was heated at 80° C. for 5 h. Additional triethyl orthoformate (0.07 mL) was added and the reaction was held at 80° C. for an additional 17 h forming a white slurry. The reaction was allowed to cool to room temperature and the solids were isolated by filtration giving 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (94.3 mg, 0.181 mmol, 35.5% yield). Analytical data matched that of the product prepared in 1A.

1C: To 1-(3-(5-(4-cyanophenoxy)pyridin-2-yl)-2-(2,4-difluorophenyl)-3,3-difluoro-2-hydroxypropyl)hydrazine-1-carbothioamide (0.5 g, 1.017 mmol) was added trimethyl orthoformate (2.25 mL, 20.35 mmol) and the reaction was heated at 95° C. for 6 h. The reaction mixture was directly loaded onto a silica gel cartridge and purified by normal phase chromatography (0-55% EtOAc/hexanes). Product containing fractions were collected and concentrated giving 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile as a white foam (380 mg, 0.606 mmol, 59.6% yield). Analytical data matched that of the product prepared in 1A.

1D: To 1-(3-(5-(4-cyanophenoxy)pyridin-2-yl)-2-(2,4-difluorophenyl)-3,3-difluoro-2-hydroxypropyl)hydrazine-1-carbothioamide (0.5 g, 1.017 mmol) was added triisopropyl orthoformate (2.27 mL, 10.17 mmol) and the reaction was heated at 100° C. for 17 h. The reaction was directly loaded onto a silica gel cartridge and purified by normal phase chromatography (0-55% EtOAc/hexanes). Product containing fractions were collected and concentrated giving 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (400 mg, 0.798 mmol, 78% yield). Analytical data matched that of the product prepared in 1A.

1E: To 1-(3-(5-(4-cyanophenoxy)pyridin-2-yl)-2-(2,4-difluorophenyl)-3,3-difluoro-2-hydroxypropyl)hydrazine-1-carbothioamide (0.5 g, 1.017 mmol) was added tripropyl orthoformate (2.19 mL, 10.17 mmol) and the reaction was heated at 100° C. for 2 h. The reaction was directly loaded onto a silica gel cartridge and purified by normal phase chromatography (0-55% EtOAc/hexanes). Product containing fractions were collected and concentrated giving 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (350 mg, 0.689 mmol, 68.6% yield). Analytical data matched that of the product prepared in 1A.

The process exemplified in Example 1 may be conducted between about 50° C. and about 150° C. with trialkyl orthoformates selected from triethyl orthoformate, trimethyl orthoformate, tripropyl orthoformate, and triisopropyl orthoformate.

Solvents that may be used in this process step include, but are not limited to, tetrahydrofuran (THF), toluene, cyclopentyl methyl ether (CPME), acetonitrile, and mixtures thereof. In some embodiments the process may be conducted in the neat trialkyl orthoformate without use of an added solvent.

1-(3-(5-(4-Cyanophenoxy)pyridin-2-yl)-2-(2,4-difluorophenyl)-3,3-difluoro-2-hydroxypropyl)hydrazine-1-carbothioamide (II) may be prepared from t-butyl 2-carbamothioyl-2-(3-(5-(4-cyanophenoxy)pyridin-2-yl)-2-(2,4-difluorophenyl)-3,3-difluoro-2-hydroxypropyl)hydrazine-1-carboxylate (III) as shown in Example 2.

### Example 2: Preparation of 1-(3-(5-(4-cyanophenoxy)pyridin-2-yl)-2-(2,4-difluorophenyl)-3,3-difluoro-2-hydroxypropyl)hydrazine-1-carbothioamide (II)

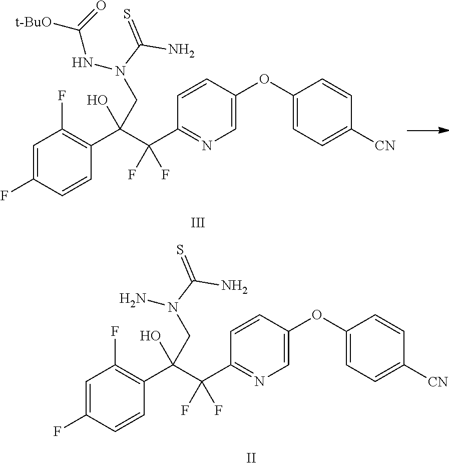

To a slurry of t-butyl 2-carbamothioyl-2-(3-(5-(4-cyanophenoxy)pyridin-2-yl)-2-(2,4-difluorophenyl)-3,3-difluoro-2-hydroxypropyl)hydrazine-1-carboxylate (III; 2 g, 3.38 mmol) in methanol (16.90 mL) was added hydrogen chloride (4M in dioxane; 4.23 mL, 16.90 mmol) and the reaction was heated at 50° C. for 1 h, after which the reaction was quenched with saturated sodium bicarbonate and extracted with ethyl acetate. The organic layer was washed with water and brine, dried over anhydrous sodium sulfate, filtered, and concentrated giving a yellow foam (1.65 g). The crude material was dissolved in dichloromethane and purified by silica gel column chromatography using 0-40% ethyl acetate/hexanes as the eluent. Product containing fractions were collected and concentrated giving 1-(3-(5-(4-cyanophenoxy)pyridin-2-yl)-2-(2,4-difluorophenyl)-3,3-difluoro-2-hydroxypropyl)hydrazine-1-carbothioamide (II) as a white foam (1.38 g, 2.81 mmol, 83% yield). ¹H NMR (400 MHz, CDCl₃) δ 8.33 (d, J=2.7 Hz, 1H), 7.74-7.62 (m, 3H), 7.56 (d, J=8.6 Hz, 1H), 7.39 (dd, J=8.7, 2.7 Hz, 1H), 7.08-7.00 (m, 2H), 6.90-6.75 (m, 2H), 6.49 (s, 1H), 5.25 (d, J=15.5 Hz, 1H), 4.89 (d, J=15.5 Hz, 1H). ¹⁹F NMR (376 MHz, CDCl₃) δ −104.41 (t, J=26.1 Hz), −108.49 (d, J=77.7 Hz), −109.07 (d, J=8.9 Hz). ESIMS m/z 492.1 [(M+H)⁺].

The process exemplified in Example 2 may be conducted between about 25° C. and about 75° C. with an acid selected from the group including hydrochloric acid (HCl), hydrobromic acid (HBr), trifluoroacetic acid (TFA), and sulfuric acid (H₂SO₄).

Solvents that may be used in this process step include, but are not limited to, methanol, ethanol, isopropanol, tetrahydrofuran, dioxane, and mixtures thereof.

t-Butyl 2-carbamothioyl-2-(3-(5-(4-cyanophenoxy)pyridin-2-yl)-2-(2,4-difluorophenyl)-3,3-difluoro-2-hydroxypropyl)hydrazine-1-carboxylate (III) may be prepared from t-butyl 2-(3-(5-(4-cyanophenoxy)pyridin-2-yl)-2-(2,4-difluorophenyl)-3,3-difluoro-2-hydroxypropyl)hydrazine-1-carboxylate (IV) as shown in Example 3.

### Example 3: Preparation of t-butyl 2-carbamothioyl-2-(3-(5-(4-cyanophenoxy)pyridin-2-yl)-2-(2,4-difluorophenyl)-3,3-difluoro-2-hydroxypropyl)hydrazine-1-carboxylate (III)

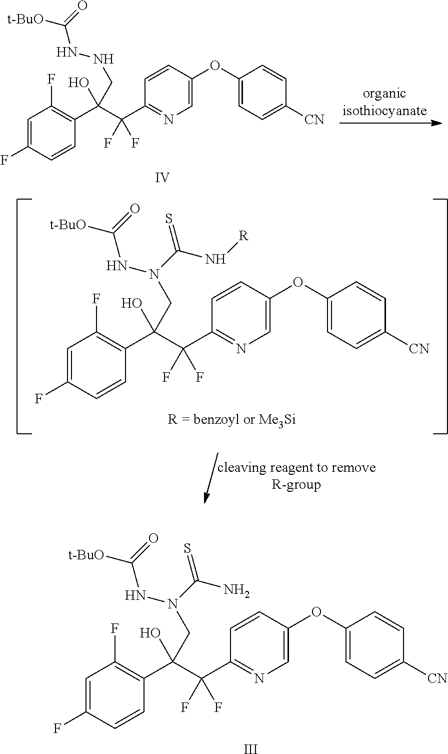

Method A: To t-butyl 2-(3-(5-(4-cyanophenoxy)pyridin-2-yl)-2-(2,4-difluorophenyl)-3,3-difluoro-2-hydroxypropyl)hydrazine-1-carboxylate (IV) (5 g, 9.39 mmol) in THF (31.3 mL) at 0° C. was added benzoyl isothiocyanate (1.199 mL, 8.92 mmol). After 30 min additional benzoyl isothiocyanate (0.1 mL, 0.74 mmol) was added. The benzoyl intermediate was identified by LCMS (ESIMS m/z 696.1 [(M+H)⁺]). After an additional 30 min, anhydrous hydrazine (1.47 mL, 46.9 mmol) was added. The mixture was stirred at 0° C. for 1 h then room temperature for 30 min. The reaction was diluted with ethyl acetate and washed with sat. ammonium chloride. The organic layer was dried over anhydrous sodium sulfate, filtered, and concentrated to a pale yellow oil. Methanol (25 mL) was added to the oil and after a few minutes of stirring a white precipitate had formed. The slurry was filtered and the solid rinsed with methanol giving t-butyl 2-carbamothioyl-2-(3-(5-(4-cyanophenoxy)pyridin-2-yl)-2-(2,4-difluorophenyl)-3,3-difluoro-2-hydroxypropyl)hydrazine-1-carboxylate (III) (4.29 g, 7.25 mmol, 77% yield) as a white solid. ¹H NMR (400 MHz, DMSO-d₆) δ 8.76 (s, 1H), 8.45 (d, J=11.9 Hz, 2H), 7.96-7.86 (m, 2H), 7.70 (dd, J=8.6, 2.8 Hz, 2H), 7.58 (d, J=8.4 Hz, 1H), 7.53-7.40 (m, 1H), 7.22-7.15 (m, 2H), 7.12 (t, J=11.0 Hz, 1H), 7.01 (d, J=8.8 Hz, 1H), 6.37 (s, 1H), 5.45 (d, J=15.7 Hz, 1H), 4.47 (d, J=15.3 Hz, 1H), 1.40 (s, 9H). ¹⁹F NMR (376 MHz, DMSO-d₆) δ−104.72 (d, J=122.8 Hz), −107.49-−109.12 (m), −111.08-−111.85 (m). ESIMS m/z 592.2 [(M+H)⁺].

Method B: To a solution of t-butyl 2-(3-(5-(4-cyanophenoxy)pyridin-2-yl)-2-(2,4-difluorophenyl)-3,3-difluoro-2-hydroxypropyl)hydrazine-1-carboxylate (IV, 1 g, 1.596 mmol) in ethyl acetate (9.4 mL) was added isothiocyanatotrimethylsilane (0.540 mL, 3.83 mmol) and the reaction was stirred at 80° C. for 18 h. NMR indicated incomplete conversion so additional isothiocyanatotrimethylsilane (0.540 mL, 3.83 mmol) was added and the reaction stirred at 80° C. for 6 h. NMR indication the reaction was still incomplete so more isothiocyanatotrimethylsilane (0.540 mL, 3.83 mmol) was added and the reaction stirred at 80° C. for 17 h. The reaction was allowed to cool to room temperature and 1 N HCl (10 mL) was added. The phases were separated and the organic layer was dried over anhydrous sodium sulfate, filtered, and concentrated to a yellow foam. The yellow foam was dissolved in methylene chloride and purified by silica gel column chromatography eluting with 0-60% ethyl acetate/hexanes. Product containing fractions were collected and concentrated giving t-butyl 2-carbamothioyl-2-(3-(5-(4-cyanophenoxy)pyridin-2-yl)-2-(2,4-difluorophenyl)-3,3-difluoro-2-hydroxypropyl)hydrazine-1-carboxylate (III) as a yellow foam (460 mg, 0.778 mmol, 49% yield). Analytical data was consistent with that of previously obtained samples.

Organic isothiocyanates for use in this process step may include acyl isothiocyanates such as, for example, benzoyl isothiocyanate and silyl isothiocyanates such as, for example, trimethylsilyl isothiocyanate.

Cleaving reagents used to remove the R-group, wherein R is benzoyl, from the unisolated intermediate to prepare the compound of Formula III, may be selected from the group including hydrazine, ammonia, sodium methoxide, and methylamine. Cleaving reagents used to remove the R-group, wherein R is trimethylsilyl, from the unisolated intermediate to prepare the compound of Formula III, may be selected from: a) fluoride compounds such as, for example, a tetraalkylammonium fluoride and potassium fluoride, and b) an acid such as, for example, hydrochloric acid (HCl), hydrobromic acid (HBr), or sulfuric acid (H₂SO₄).

The contacting of the compound of Formula IV with the organic isothiocyanate may be carried out between about −20° C. and about 100° C., and the contacting with the cleaving reagent may be carried out between about −20° C. and about 100° C.

Solvents for use in this process step may include one or more than one of THF (tetrahydrofuran), EtOAc, 2-Me-THF, dioxane, MeCN (acetonitrile), and DME (1,2-dimethoxyethane).

t-Butyl 2-(3-(5-(4-cyanophenoxy)pyridin-2-yl)-2-(2,4-difluorophenyl)-3,3-difluoro-2-hydroxypropyl)hydrazine-1-carboxylate (II) may be prepared from 4-((6-((2-(2,4-difluorophenyl)oxiran-2-yl)difluoromethyl)pyridin-3-yl)oxy)benzonitrile (III) as shown in Example 2.

### Example 4: Preparation of t-butyl 2-(3-(5-(4-cyanophenoxy)pyridin-2-yl)-2-(2,4-difluorophenyl)-3,3-difluoro-2-hydroxypropyl)hydrazine-1-carboxylate (IV)

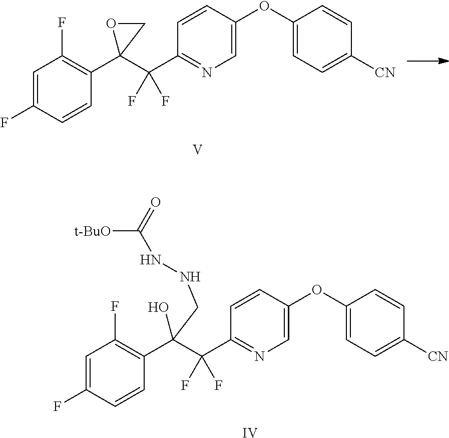

To a slurry of 4-((6-((2-(2,4-difluorophenyl)oxiran-2-yl)difluoromethyl)pyridin-3-yl)oxy)benzonitrile (V) (5 g, 12.49 mmol) in ethanol (40 mL) was added t-butyl carbazate (4.13 g, 31.2 mmol) and the reaction was heated at 80° C. for 24 h, at which point the starting epoxide (V) was completely consumed. The reaction was allowed to cool to 45° C. and seeded with crystals of product IV causing the reaction to cloud. Additional ethanol (40 mL) was added, and the reaction was cooled to room temperature overnight. The resulting slurry was cooled with an ice bath for 30 min and filtered. The solids were rinsed with ethanol (30 mL) and dried under vacuum providing t-butyl 2-(3-(5-(4-cyanophenoxy)pyridin-2-yl)-2-(2,4-difluorophenyl)-3,3-difluoro-2-hydroxypropyl)hydrazine-1-carboxylate (IV) as a white solid (5.42 g, 9.67 mmol, 77% yield). ¹H NMR (400 MHz, CDCl₃) δ 8.37 (d, J=2.7 Hz, 1H), 7.72-7.64 (m, 2H), 7.55 (td, J=8.8, 6.6 Hz, 1H), 7.48 (d, J=8.6 Hz, 1H), 7.37 (dd, J=8.7, 2.7 Hz, 1H), 7.10-7.02 (m, 2H), 6.77 (dddd, J=20.9, 11.4, 8.6, 2.6 Hz, 2H), 3.83 (d, J=13.7 Hz, 1H), 3.74 (dd, J=13.4, 2.8 Hz, 1H), 1.41 (s, 9H). ¹⁹F NMR (376 MHz, CDCl₃) δ −105.15, −108.68 (d, J=22.1 Hz), −109.24, −110.29. ESIMS m/z 533.1 [(M+H)⁺].

The contacting of the compound of Formula V with t-butyl carbazate may be carried out from about 25° C. to about 100° C. or from about 60° C. to about 90° C.

Solvents for use in this process step may include alcohols such as methanol, ethanol, and isopropanol, as well as aprotic solvents such as THF (tetrahydrofuran), acetonitrile, DMSO (dimethylsulfoxide), DMF (N,N-dimethylformamide), and mixtures of any of these solvents.

What is claimed is:

1. A method of making a compound of Formula I comprising: 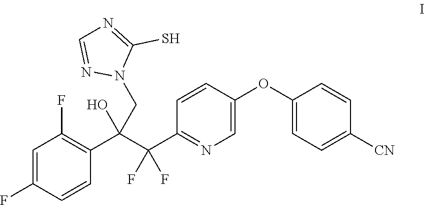 [SMILES: N#Cc1ccc(Oc2ccc(C(F)(F)C(O)(Cn3ncnc3S)c3ccc(F)cc3F)nc2)cc1]
contacting a compound of Formula II 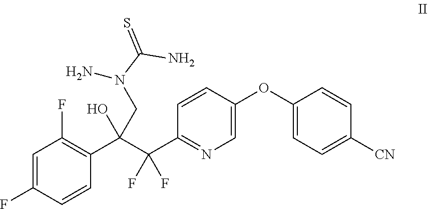 [SMILES: N#Cc1ccc(Oc2ccc(C(F)(F)C(O)(CN(N)C(N)=S)c3ccc(F)cc3F)nc2)cc1]
with a trialkyl orthoformate.

2. The method of claim 1, wherein the trialkyl orthoformate is selected from the group including triethyl orthoformate, trimethyl orthoformate, tripropyl orthoformate, and triisopropyl orthoformate.

3. The method of claim 1, wherein the contacting is carried out between about 50° C. and about 150° C.

4. The method of claim 1 further comprising a solvent selected from the group including tetrahydrofuran, toluene, cyclopentyl methyl ether, acetonitrile, and mixtures thereof.

5. The method of claim 1 further comprising the step of contacting a compound of Formula III: 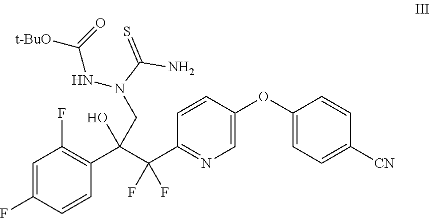 [SMILES: CC(C)(C)OC(=O)NN(CC(O)(c1ccc(F)cc1F)C(F)(F)c1ccc(Oc2ccc(C#N)cc2)cn1)C(N)=S]
with an acid to prepare the compound of Formula II.

6. The method of claim 5 wherein the acid is selected from the group including HCl, HBr, TFA, and H₂SO₄.

7. The method of claim 5 wherein the contacting is carried out between about 25° C. and about 75° C.

8. The method of claim 5 further comprising a solvent selected from the group including methanol, ethanol, isopropanol, tetrahydrofuran, dioxane, and mixtures thereof.

9. A compound consisting of: 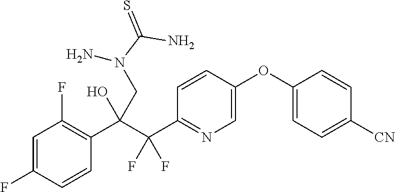 [SMILES: N#Cc1ccc(Oc2ccc(C(F)(F)C(O)(CN(N)C(N)=S)c3ccc(F)cc3F)nc2)cc1]
